(19) United States
(12) Patent Application Publication
(10) Pub. No.: US 2009/0209955 A1
(43) Pub. Date: Aug. 20, 2009

(54) PROSTHETIC VALVE IMPLANT SITE PREPARATION TECHNIQUES

(76) Inventors: David C. Forster, Los Altos Hills, CA (US); Brian Beckey, Woodside, CA (US); Brandon Walsh, Syracuse, UT (US); Scott Heneveld, Whitmore, CA (US)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(21) Appl. No.: 12/305,611

(22) PCT Filed: Jun. 20, 2007
(86) PCT No.: PCT/US07/71646

§ 371 Date: Dec. 18, 2008

Related U.S. Application Data
(60) Provisional application No. 60/805,333, filed on Jun. 20, 2006.

(51) Int. Cl. A61B 18/18; A61N 7/00; A61B 17/32; A61B 17/10; A61M 29/02

(52) U.S. Cl. 606/33; 601/2; 606/170; 606/140; 606/192

(57) ABSTRACT

Prosthetic valves implantation methods and systems, especially as related to preparing the native site of a native stenotic or incompetent aortic valve for receipt of a prosthetic replacement valve are described. The subject tools and associated site preparation techniques may be employed in percutaneous aortic valve replacement procedures.

### BACKGROUND

Diseases and other disorders of the heart valves affect the proper flow of blood from the heart. Two categories of heart valve disease are stenosis and incompetence. Stenosis refers to a failure of the valve to open fully, due to stiffened valve tissue. Incompetence refers to valves that cause inefficient blood circulation by permitting backflow of blood in the heart.

Medication may be used to treat some heart valve disorders, but many cases require replacement of the native valve with a prosthetic heart valve. Prosthetic heart valves can be used to replace any of the native heart valves (aortic, mitral, tricuspid or pulmonary), although repair or replacement of the aortic or mitral valves is most common because they reside in the left side of the heart where pressures are the greatest.

Conventional heart valve replacement surgery involves accessing the heart in the patient's thoracic cavity through a longitudinal incision in the chest. For example, a median sternotomy requires cutting through the sternum and forcing the two opposing halves of the rib cage to be spread apart, allowing access to the thoracic cavity and heart within. The patient is then placed on cardiopulmonary bypass support which involves stopping the heart to permit access to the internal chambers. Such open heart surgery is particularly invasive and involves a lengthy and difficult recovery period.

Percutaneous implantation of a prosthetic valve is a preferred procedure because the operation is performed under local anesthesia, may not require cardiopulmonary bypass, and is less traumatic. Various types of prosthetics are adapted for such use. One class employs a stent like outer body and internal valve leaflets attached thereto to provide one way blood flow. These stent structures are radially contracted for delivery to the intended site, and then expanded/deployed to achieve a tubular structure in the annulus. Another more advantageous class is offered by the assignee hereof US Patent Publication No. 2005-0203614 (which application is incorporated by reference herein in its entirety) describes a system in which various panels define the implant body carrying valve leaflets. These prosthetic valve structures are delivered in a contracted state and then unfolded and/or unrolled into an expanded state at the treatment location.

With either type of structure, a sufficient engagement between patient body tissue and the prosthesis body is desired to secure the position of the implant and form a peripheral seal. However, when implanting the prosthetic device at the site of/within the envelope of the native valve, the condition of the native valve can interfere with fit. Stated otherwise, irregularity in the shape of the implantation site, surface features, texture, and composition pose challenges for developing an implant of a regular size able to accommodate all such variability.

Aspects of the invention optionally address the challenges presented by prosthetic member interface with calcific and/or irregular valve leaflet and annulus geometry. In addition, other advantages of the present invention may be apparent to those with skill in the art upon review of the subject disclosure.

### SUMMARY

Described herein are systems and methods for the preparation of target tissue in anticipation of the implantation of a prosthesis. Devices for modification or removal of tissue through chemical techniques, mechanical techniques and the application of energy are described. Devices for creating treatment zones and other devices are also described.

### DRAWINGS

The figures provided herein are not necessarily drawn to scale, with some components and features being exaggerated for clarity. Each of the figures diagrammatically illustrates aspects of the systems and methods described herein. Variation from the embodiments pictured is fully contemplated.

FIGS. 1A-B are end on views depicting exemplary embodiments of a cutting device.

FIGS. 1C-D are perspective views depicting additional exemplary embodiments of a cutting device.

FIGS. 2A-3C are perspective views depicting additional exemplary embodiments of a tissue modifying device.

FIG. 4A is a perspective view depicting an exemplary embodiment of a treatment zone creation device.

FIG. 4B is a cross-sectional view depicting another exemplary embodiment of a treatment zone creation device.

FIGS. 5A-D are perspective views depicting an exemplary embodiment of a cutting device.

FIGS. 5E-G are top down views depicting an exemplary embodiment of a cutting device.

FIGS. 6A-E are perspective views depicting additional exemplary embodiments of a cutting device.

FIGS. 7A-B are side views depicting another exemplary embodiment of a cutting device.

### DETAILED DESCRIPTION OF THE INVENTION

Various exemplary embodiments of the invention are described below. Reference is made to these examples in a non-limiting sense. They are provided to illustrate more broadly applicable aspects of the invention. Various changes may be made to the systems and methods described and equivalents may be substituted without departing from the true spirit and scope of the inventions. In addition, many modifications may be made to adapt a particular situation, material, composition of matter, process, process act(s) or step(s) to the objective(s), spirit or scope of the present inventions. Further, as will be appreciated by those with skill in the art that each of the individual variations described and illustrated herein has discrete components and features which may be readily separated from or combined with the features of any of the other several embodiments without departing from the scope or spirit of the present inventions. All such modifications are intended to be within the scope of the appended claims.

Accordingly, methods described herein include methods of prosthetic valve site preparation to homogenize, smooth and/or make the site more regular. These methods are typically (most advantageously, though not necessarily) performed percutaneously. Any number of the techniques described or some combination of them are employed to modify the tissue (the valve segments themselves and/or surrounding tissue) for forming an improved tissue-valve body interface with prosthetic to be implanted.

Prior to any such removal and/or tissue manipulation alone, it may be desirable to perform balloon valvuloplasty upon the selected valve. In the case of an aortic valve, the valve leaflet(s) are at least partially opened by dilating a balloon after crossing the valve with a guidewire and passing the balloon over the wire to the treatment site. The valvuloplasty procedure may be performed using a conventional balloon or a cutting balloon adapted to cut the leaflets so that they open more easily.

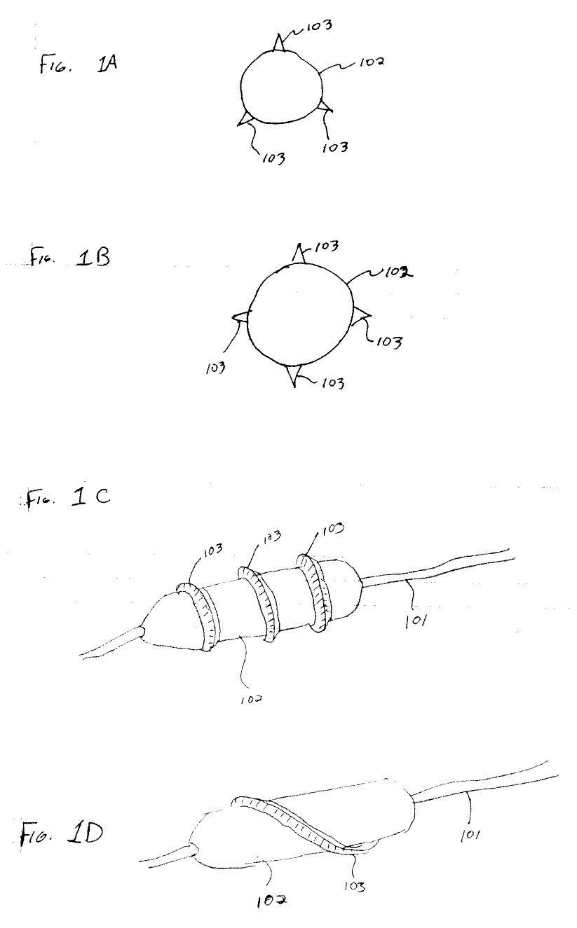

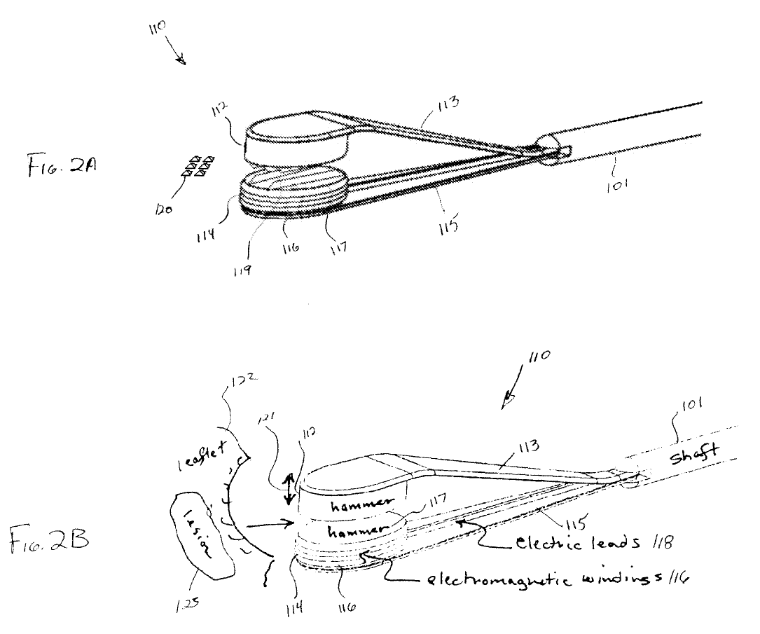

FIGS. 1A-D depict exemplary embodiments of a cutting balloon 102 located on an elongate shaft 101. FIGS. 1A-B are end on views of balloon 102 in an inflated state with various configurations of multiple longitudinally aligned cutting elements 103. FIGS. 1C-D are perspective views depicting alternative arrangements of the cutting elements 103. In FIG. 1C, multiple cutting elements 103 are present and are aligned radially about balloon 102. In FIG. 1D, one cutting element is shown disposed both longitudinally and radially along the surface of balloon 102.

U.S. Publication No. 2006/0116700 (incorporated herein by reference in its entirety) provides another example of a cutting balloon as may be used with the systems and methods described herein. Other cutting balloons that may be adapted for such use include the Flextome Cutting Balloon® (Boston Scientific) and those described in U.S. Pat. Nos. 5,196,024; 6,632,231 and 6,951,566 as well as in US Publication No. 2005/002107 (wherein each of these references are also incorporated by reference in their entireties).

Aside from such pre-treatment options, in one exemplary embodiment, a percutaneous approach for physical manipulation of the tissue is provided. In another exemplary embodiment, the chemistry of the site is modified. In others, energy is applied to modify the tissue.

The intended result of such manipulation may be to simply provide a more compliant or malleable site for prosthetic implantation that is better able to accommodate (e.g., conform to) implant geometry for the purpose of retention, accuracy in placement, sealing, etc. (even if the tissue subsequently remodels). Otherwise, the tissue softening achieved may be for the purpose of assisting in removal of at least some of the tissue. Such removal may be desired in order further improve on such advances possible with a tissue modification approach, alone.

As for mechanical methods for tissue modification, one or more tools are provided to homogenize, break-up, etc. stenotic valve leaflet tissue. Calcium nodules or deposits within the tissue are broken-up or broken-down by physical action. By such physical or mechanical action, what is meant is that the tissue acted upon is hit or struck by or between bodies adapted for such use. In one exemplary embodiment using a hammer-and-anvil type approach, stiffened tissue is made more compliant by disrupting its (typically) calcified structure.

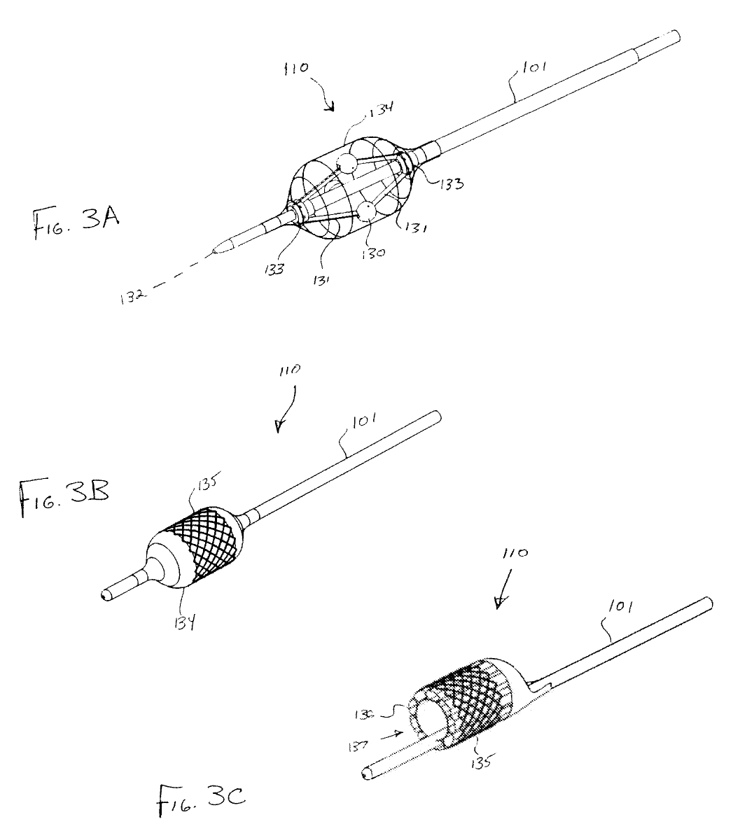

FIGS. 2A-B are perspective views depicting an exemplary embodiment of a device 110 suitable for such use having opposing bodies 112 and 114 that are brought together at speed with sufficient kinetic energy to modify the problematic tissue. In one variation as shown here, opposing hammer bodies 112 and 114 (or hammer-and-anvil mass elements) are mounted on struts or arms 113 and 115, which are in turn coupled to elongate shaft 101. In another variation, blades or spike-like features may be mounted on the bodies (as shown in FIG. 2A), or used alone (as shown in FIG. 2B). The arms may be biased apart as shown in FIGS. 2A-B. Preferably, one of the bodies comprises a permanent magnet (e.g., a rare-earth magnet) or is ferromagnetic. In either case, operation of an opposite-facing electromagnetic body attracts the other along direction 121.

In FIGS. 2A-B, hammer body 112 is ferromagnetic and hammer body 114 is configured as an electromagnet with electromagnetic windings 116 wrapped about a ferromagnetic center portion 117. Windings 116 are coupled with electric leads 118 disposed along arm 115. Leads 118 are in turn coupled with a power source such that the appropriate current can be applied to windings 116 to generate the electromagnetic field from hammer 114. When a magnet is used opposite the electromagnet, switching polarity of the electromagnet can also drive the bodies apart along direction 121. Otherwise, spring force is to be relied upon to keep or force the members apart to a distance after power is cut to the electromagnet so that they can strike each other in a repeated fashion. FIG. 2B depicts device 110 prior to application to calcified lesion 123 in leaflet 122.

The striking action may be purely user-directed, or cyclic once initiated by user input. It may be continuous until terminated by a user, or some number of cycles may automatically proceed once triggered by a medical practitioner. For example, the hammering mechanism may repeat 10, 20, 50 or 100 times in one “shot.” For any such cyclic or repetitive motion, it may progress at a rate between 1 and 1000 Hz. More typically, it is in the range of about 10 to about 100 Hz. Such action may be controlled by switching DC voltage (e.g., in the example of the spring-biased approach) or by applying AC voltage (e.g., in the example where a permanent magnet is used).

Advantageously, the opening and closing of the bodies is such (i.e., the “jaws” of the device open wide enough and for so long a period of time) that the device can be navigated or moved between contiguous tissue sites during operation. Alternatively, the method may progress by treating one site, initiating device action to modify the target tissue, waiting for device action termination and then repeating such action in succession. The striking bodies will typically be held together by attraction during delivery.

In another approach to breaking-up or otherwise modifying calcific lesions involves striking them with an expandable set of spinning weights. The weights may be carried on flexible or articulating linkage arms or arranged otherwise, such as that depicted in the exemplary embodiment of device 110 shown in the perspective view of FIG. 3A. Here, weights 130 are pivotably coupled to linkage arms 131, which are turn pivotably coupled to hubs 133. Hubs 133 are in turn rotatably coupled to elongate shaft 101 and one or more of hubs 133 are also slidably coupled with shaft 101. Device 110 is configured to rotate weights 130 about axis 132. Rotation of the bodies about an axis can be used to expand the radius of the arc along which they travel. In the embodiment of FIG. 3A, the rate of rotation of weights 130 causes linkage arms 131 to pivot with respect to hubs 133 and weights 130, and also causes one or both of hubs 133 to slide towards weights 130, to expand the arc along which weights 130 travel. Weights 130 may be circumscribed or surrounded by an expandable sheath or balloon 134 (the outline of which is shown in FIG. 3A to allow visibility to the components within).

Either way, such a covering 134 may keep the weights from fouling the target tissue or becoming entangled and stripping or ripping off loose material. Use of balloon 134 may be desirable for (when inflated) centering the action of device 110 within the aorta lumen or valve annulus. The rate at which weights 131 spin may vary widely, the optimization of which will depend on the design selected.

FIG. 3B is a perspective view depicting another exemplary embodiment of device 110. Here, the components within balloon 134 are not shown for clarity. In this embodiment, balloon 134 is shown covered with an additional abrasive covering 135. Here, abrasive covering 135 has a mesh-like configuration configured to both abrade the target tissue during rotation of weights 130 and provide reinforcement to balloon 134 in the event that balloon 134 is susceptible to rupture by weights 130.

FIG. 3C is a perspective view depicting another exemplary embodiment of device 110. In this embodiment, device 110 includes a distal portion 137 having multiple weights 130 arranged in an eccentric, cylindrical configuration about shaft 101. Distal portion 137 has a center of mass offset from elongate shaft 101. Rotation of shaft 101 causes portion 137 to oscillate and strike and preferably modify the outlying target tissue.

Any of these mechanical systems that may be used in mechanical tissue modification methods typically include an operative or working end of the device, a medial shaft or catheter body leading thereto (including any retractable sheath portion offered to cover or secure the device when navigating tortuous anatomy) and an integrated or separate/reusable power supplies and/or electronic controls.

As referenced above, in another exemplary embodiment, tissue is modified by chemical means. Chemical modification of aortic valves including their leaflets is taught in various patents by Constantz et al. (e.g., U.S. Pat. Nos. 6,712,798; 6,622,732; 6,533,767; 6,394,096; 6,387,071 and 6,379,345—each incorporated by reference herein in its entirety). They variously teach valve and/or annuli demineralization by application of a low pH solution for a period of time.

The treatments referenced in the patents include demineralizing valvuloplasties or annuloplasties. As described, in demineralizing a valve/annular structure the valve or structure having the calcified lesion present thereon, the site is typically flushed with a dissolution solution. Two demineralizing acid solutions of particular interest noted are hydrogen chloride solutions and carbonic acid solutions. The dissolution solutions employed may also comprise one or more additional components that serve a variety of purposes as taught in the referenced patents. While such teachings are fully applicable to carrying out the systems and methods described herein, different applications for the demineralizing technology beyond use for valvuloplasties and annuloplasties are also contemplated.

Specifically, in another exemplary method, demineralization techniques are employed to change the compliance of a bulk area adjacent to and including the valve to provide an improved interface for a luminal implant delivered percutaneously and situated at or adjacent to the demineralization region. The additional compliance gained by the lumen wall and/or native valve (leaflets and/or annulus) can dramatically assist in forming a patent seal with the implant and help avoid device migration.

Accordingly, while the systems and methods described herein may employ the specific solutions and some of the techniques described in any of the Constantz patents, the methods herein differ in at least that such action is followed by other acts. Namely, the chemical tissue modification may precede tissue removal. Alternatively (or additionally), the chemical tissue modification may offer critical preparation of the implantation site from the perspective of providing a suitable level of tissue conformability to or with the implant to be delivered, especially percutaneously.

Still further, unique devices are provided for application of the acidic dissolution medium. One such device includes a lumen for blood to pass through and a pair of annular balloon used to define a solution chamber including the aorta wall. The length of the chamber formed (and hence the device itself) is coordinated for use with a selected aortic valve implant. The length of the lumen segment modified for increased compliance may substantially correspond to the length of the prosthesis in contact with the wall. Alternatively, a longer or shorter section could be modified. A longer section may allow the implant to better embed in surrounding tissue, allowing end captured. A shorter section may be desired to minimize vessel trauma, or simply to target any engagement features provided on the prosthesis body-alone.

In yet another exemplary method for modifying calcific tissue for implant-tissue interface improvement or ease of resection to assist in implant receipt, thermal energy is applied to soften calcific lesions, especially to those in the leaflets. One approach to applying such energy is by a stent-like body or coil pattern carried by or imprinted on the body of a balloon. Once at the treatment site (such as at the aortic valve annulus), the balloon is inflated to hold the metallic structure in contact with tissue. Radio frequency (RF) or microwave energy can be delivered by the device. Alternatively, ohmic/resistive heating may be employed to heat the metallic body directly. The balloon may be a single-chamber device or a multi-chamber device. The devices may be configured in single-chamber toroidal form or configured to provide a central lumen using multiple radially-oriented chambers.

In another energy-delivery approach, a treatment zone is created by use of one or more balloons and/or baffle wall(s) to form a working region along a vessel wall that is evacuated of blood. So prepared, laser energy—delivered locally by one or more diodes, or transmitted along one or more fiber optics—is applied to alter/modify calcific tissue or selectively ablate tissue as desired for more aggressive site modification.

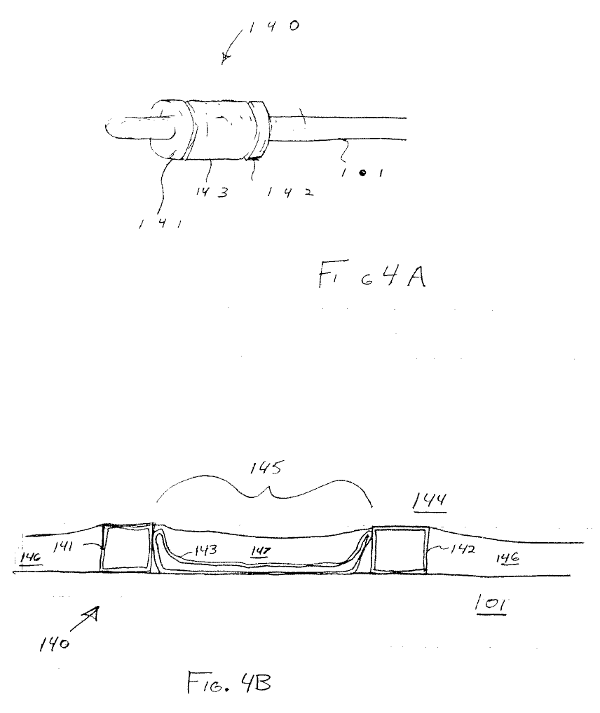

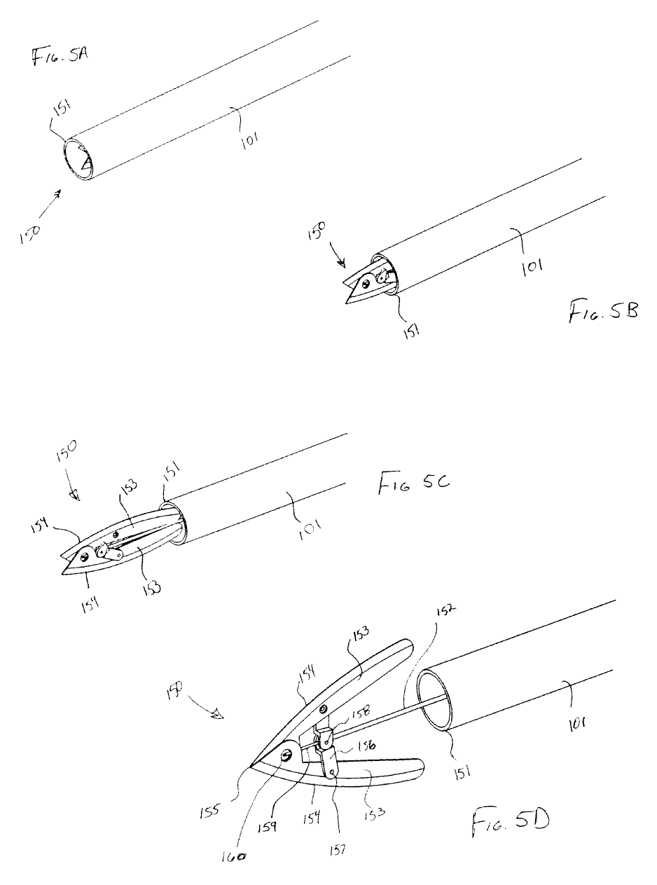

FIGS. 4A-B depict exemplary embodiments of a device 140 configured to generate a treatment zone. FIG. 4A is a perspective view depicting device 140 with three expandable membranes or balloons 141-143 located on elongate shaft 101 in their expanded states. Each balloon 141-143 has a toroidal configuration. Zone creation balloon 143 is configured to expand into contact with the surrounding vessel wall or tissue and evacuate any intervening fluids away from the vessel wall/tissue.

FIG. 4B is a cross-sectional view depicting this embodiment after creation of a treatment zone 145. Here, zone 145 preferably corresponds to the presence of target tissue, e.g., calcified tissue, in a vessel wall 144. After inflation of zone creation balloon 143, end cap balloons 141 and 142 are inflated to create a barrier to the entrance of surrounding fluids 146. Zone creation balloon 143 can then be deflated to create a treatment space 147 conducive to the use of a treatment, such as an energy application apparatus (e.g., a laser). End cap balloons 141 and 142 are preferably strong enough to resist the passage of fluids into the treatment space 147. A suction-irrigation port (not shown) can also be included to remove ablated tissue or by-products of the treatment (e.g., smoke) and the like. The inflation lumens for the balloons and any blood shunting lumen(s) are not shown for clarity.

In still another exemplary energy-delivery approach, lithotripsy techniques involving shock waves are employed. Such technology is typically used to break up “stones” that form in the kidneys, bladder, ureters or gallbladder. Though there are multiple approaches which may be employed, the most common approach is extracorporeal shock wave lithotripsy. The shock waves are focused at the treatment site to break the calcific bodies into tiny pieces. In the case of kidney stones, the patient “passes” them. When practicing the technique on calcified leaflet lesions according to the present invention, no passage of the nodules is either required, nor is there a pathway for such action. Accordingly, it can be practiced without need for embolic protection or the like due to the encapsulated nature of aortic valve mineralization.

Most preferably, all these methods and combined permutations thereof are performed in the context of a beating-heart procedure. In other words, the patient need not be placed on cardiopulmonary bypass support during the procedure. However, aspects of the subject method and tools described herein may be employed under such conditions.

Another contemplated variation of the systems and methods described herein concerns the removal of certain tissues. Indeed, the tissue modification described may serve as a precursor to tissue removal. So-modified, tissue removal using conventional and/or modified techniques may be simplified. For such purposes, any of the following devices and/or techniques (and still others) may be employed:

Radiofrequency (RF) Ablation

Generally, by way of a non-limiting example, monopolar radiofrequency thermal ablation transfers radiofrequency energy to the leaflet tissue through probes inserted in the leaflet. The energy can raise the temperature of the tissue, thus using thermal injury to transect surrounding tissue. A secondary feature is used to remove the free tissue.

Bipolar Radiofrequency Ablation (Coblation)

Generally, by way of a non-limiting example, this procedure produces an ionized saline layer that disrupts molecular bonds without using heat. As the energy is transferred to the tissue, ionic dissociation occurs. This mechanism can be used to remove all or only part of the leaflet. This causes removal of tissue with a thermal effect of 45-85 C.°. The advantages of this technique are lessened trauma to adjacent tissue. A secondary feature is used to remove the dissolved tissue and residual saline plasma.

Laser

Generally, by way of a non-limiting example, this technique employs laser at the end of the catheter to vaporize and remove leaflet tissue. A secondary feature (such as graspers or down-stream filters) is used to remove the dissected tissue.

Ultrasound

Generally, by way of a non-limiting example, a mechanism is used to transfer ultrasound energy to the leaflet tissue. The energy raises the temperature of the tissue, thus using thermal injury to transect surrounding tissue. The mechanism could be a probe in close proximity to the tissue. Other device features could position the probe in intimate contact with the tissue, thus improving efficacy. Those features could include catheter steering or one or more toroidal balloons to create good apposition.

Snare Wire

Generally, by way of a non-limiting example, this technique employs a “noose shaped” snare wire oriented with the free ends running down a catheter shaft. A grasper mechanism may be incorporated with the snare to approximate large calcified tissue. After tissue is approximated, the snare wire is pulled in tension, causing the loop to closes down on the proximal tissue within its radial space. The collapsed tissue is severed. A secondary feature is used to remove the dissected tissue. Various loop-based cutter approaches and options are disclosed in the figures.

Harmonic Scalpel

Generally, by way of a non-limiting example, this device uses ultrasonic energy to vibrate its blade at, e.g., 55,000 cycles per second. Invisible to the naked eye, the vibration transfers energy to the tissue, providing simultaneous cutting and coagulation. The temperature of the surrounding tissue reaches 80 degrees Celsius. The end result is precise cutting with minimal thermal damage. The blade could be configured as a single blade (curved, straight, arrow head, etc), a dual blade shear, a guillotine type design, or some other embodiment. A secondary feature is used to remove the dissected tissue.

Window Cutters

Generally, by way of non-limiting examples, these devices often employ a cannula-type body or extension with a side-hole. Vacuum in the lumen or the pressure of impinging tissue pushes material into the side hole. It is severed by an internal or external sleeve (optionally sharpened) actuated to close-offer the side port. US Publication No. 2004/0049215 discloses such a device including an ultrasonic transducer vision system.

Artherectomy Devices

Generally, by way of non-limiting examples, a wide variety of cutter types are presented in this class. Examples of rotary cutters include the Rotoblator™ tool (Boston Scientific) and the burr presented in U.S. Pat. No. 6,503,261. Others include the SilverHawk™ system (Fox Hollow) as described in US Publication No. 2005/0222663 and those presented in U.S. Pat. Nos. 5,507,760; 5,624,457; 5,669,920 and 6,120,515. Another cutter of similar construction described in U.S. Pat. No. 5,429,136 includes a vision system. A ramp or drill type cutter including visions systems (e.g. ultrasound transducer arrays) is disclosed in U.S. Pat. No. 6,027,450. U.S. Pat. No. 5,312,425 discloses yet another type of artherectomy devise employing a turning helical blade, while U.S. Pat. No. 5,181,920 discloses a rotating scoop-type cutter. This last device and several of the others also include a balloon to assist in positioning the device and/or forcing material into contact with the cutting means.

Custom Cutters

Various other types of unique cutters produced especially for use with the systems and methods described herein may be employed. In the figures, an arrow-style cutter is shown together with its method of use. Also, a coaxial cutter that employs suction of tissue into contact with a round or elliptical blade face (that may be shielded by an atraumatic-tip external sleeve for placement/navigation) can be used. The former device offers advantages in terms of articulation and delivery of an enlarged cutting blade; the latter device is desirable in view of its elegance in design and ability to aspirate/withdraw tissue through its central lumen. Still, graspers or forceps may be used in connection with either cutter to capture or manipulate the tissue worked upon. A combination grasper/cutting jaw tissue removal is also shown.

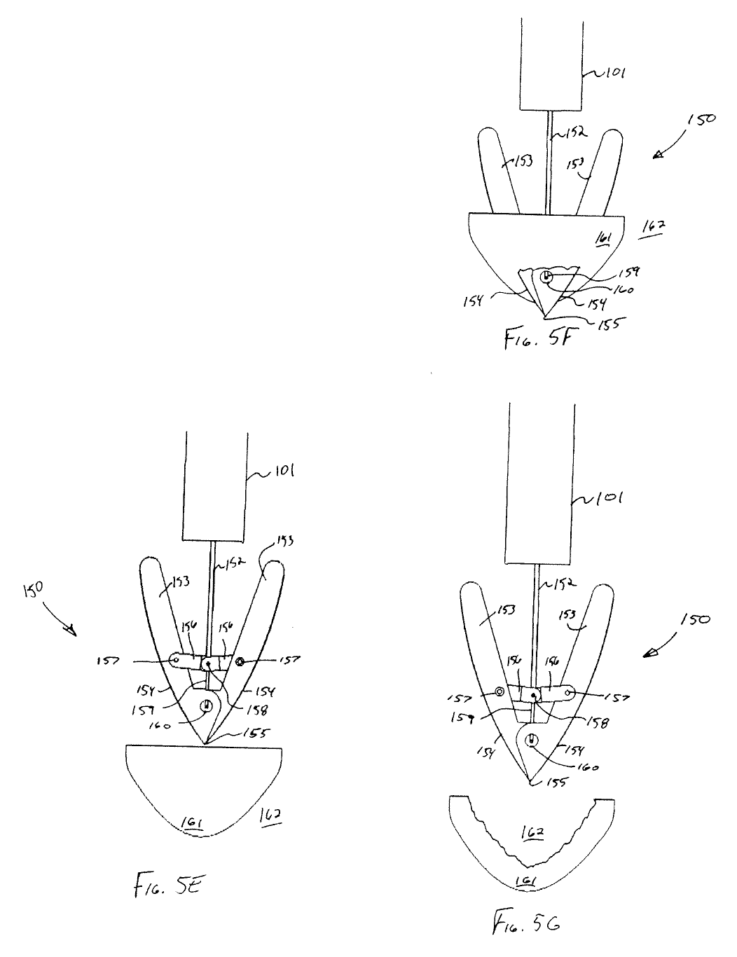

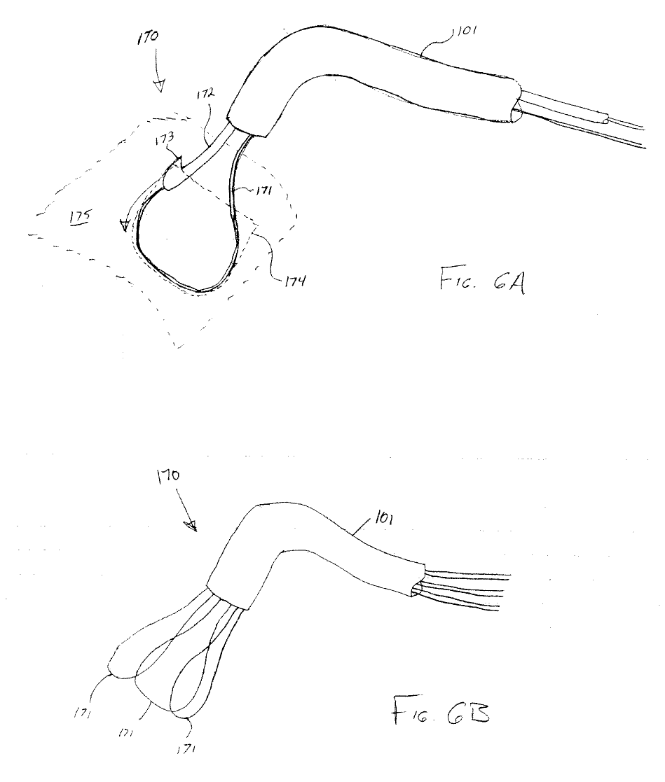

FIGS. 5A-G are perspective and top down views depicting an exemplary embodiment of an arrow-style cutter and its method of use. FIGS. 5A-D show sequential advancement of cutter 150 from elongate shaft 101 through open distal end 151. Cutter 150 is housed within shaft 101 in a compressed or folded configuration and is preferably configured to be expandable into the cutting configuration of FIG. 5D. Cutter 150 includes two blade members 153 with sharp outer edges 154. Blade members 153 are coupled to an elongate support shaft 152 having a secondary shaft 159 slidably coupled therewith, such as within an inner lumen of support shaft 152. Linkage members 156 pivotably couple each blade member 153 with support shaft 152. Here, linkage members 156 are pivotably coupled with blade member 153 at pivot 157 and with support shaft 152 at pivot 158. One or more bias members (not shown) can be used to cause blade members 153 to automatically enter the expanded configuration as shown in FIGS. 5C-D. Once in the expanded configuration, blade members 153 preferably align to form a sharp distal tip 155 as shown in FIG. 5D.

FIG. 5E depicts cutter 150 in proximity with the target tissue, which in this embodiment is a valve leaflet 161 to be removed as part of a valvuloplasty procedure. Cutter 150 can be advanced between leaflet 161 and the vessel wall 162 to cause sharp distal tip 155 to penetrate leaflet 161 as shown in FIG. 5F. Sharp outer edges 154 allow continued distal movement of cutter 150 to sever substantially all of leaflet 161, as shown in FIG. 5G. The severed tissue can then be collected using an embolic filter (not shown) as described herein. Once the procedure is complete, advancement of secondary shaft 159 against abutments 160 causes blade members 153 to deflect back into the compressed configuration to allow withdrawal of cutter 150 back into shaft 101.

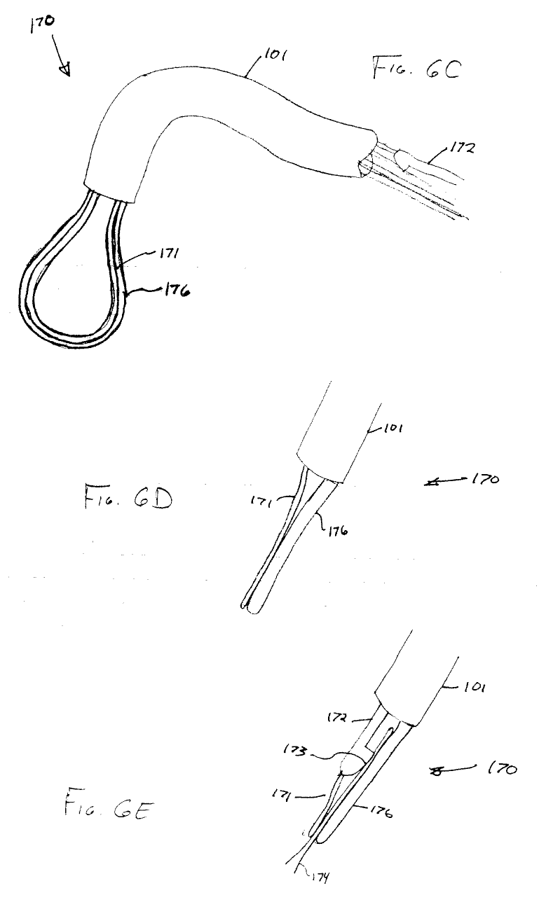

FIGS. 6A-E are perspective views depicting additional exemplary embodiments of a cutter 170 for use with the systems and methods described herein. FIG. 6A depicts device 170 having a flexible wire-like loop element 171 after advancement from within shaft 101 through open distal end 151. Element 171 is preferably configured to conform to the target tissue, which in this embodiment is valve leaflet 174. Alternatively, element 171 can have a predetermined shape for conformance with the target tissue. Element 171 is shown here located in the pocket created between leaflet 174 and vessel tissue 175. A cutting element 172, a sharp outer edge 173, is slidably disposed over wire-like element 171 such that movement of element 172 is guided along the target tissue by wire element 171. This allows sharp outer edge 173 to incise the tissue, allowing leaflet 174 to be severed.

FIG. 6B depicts another exemplary embodiment having three wire-like elements 171 allowing for multiple cutting elements 172 (not shown) to be used simultaneously in order to speed the procedure.

FIGS. 6C-D depict yet another exemplary embodiment of cutter 170. Here, a flexible wire band-like element 176 is included with wire-like element 171 and both are extendable from within shaft 101. Wire-like element 171 and band-like element 176 are preferably configured to swing in towards each other as depicted in the side view of FIG. 6D. During an exemplary procedure, such as a valvuloplasty procedure, one or the other of elements 171 and 176 is inserted into the pocket between leaflet 174 and vessel wall 175 (both not shown). The other is left outside of the pocket. Cutting element 172 can then be advanced over wire-like element 171 and used to sever leaflet 174 lying between elements 172 and 176. In this manner, elements 171 and 176 pinch the target tissue therebetween and provide enhanced control in the cutting process.

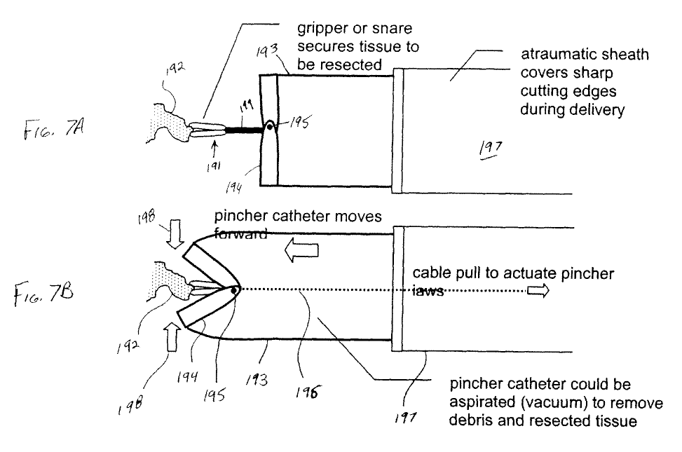

FIGS. 7A-B depict an exemplary embodiment of a cutting device 190 having a grasping device 191 for grasping and better isolating the target tissue 192. Grasping device 191 is coupled to a support shaft 199 and can be configured in a forcep-like manner, such as that shown here, or can have a snare-like configuration. Grasping device 191 and cutting jaw device 193 are moveable relative to each other, as shown in FIG. 7B, and grasping device 191 can facilitate the proper location of cutting jaw device 173 with respect to target tissue 192. Here, once cutting jaw device 193 is positioned relative to the target tissue as desired, the blade-like jaws 194 can be actuated and closed along directions 198, preferably be retraction of a pull wire 196, in order to sever the target tissue 192. An atraumatic sheath 197 can be used to slide over devices 191 and 193 and cover any potentially traumatic surfaces during delivery. Cutting jaw device 193 can also include vacuum or suction capability to withdraw any debris and severed tissue.

For the known devices, their bodies may be lengthened and/or made more flexile or torque responsive in order to facilitate navigating anatomy to reach the aortic valve site. Other adaptations of the above-referenced tools as understood by those with skill in the art may additionally or alternatively be desirable in order to improve their efficacy in the subject method. One such modification is to include a vision system integrated with the device. The “vision” system may comprise provision for ultrasonic imaging, optical fibers for optical coherence tomography, or another means. Still, an ancillary scope or vision system (e.g., as in an endoscope, IVUS catheter, etc.) as routinely used in percutaneous procedures may be employed. Of course, other variations are possible as well.

In order to make use of such devices for the purpose of tissue removal, it may additionally be desirable to employ techniques as described in US Publication No. 2005/01071472 to isolate the valve operated upon. Alternatively or additionally, leaflet removal may be accomplished employing the teachings in U.S. Pat. No. 5,295,958 and/or US Publication Nos. 2001/0044591 and 2004/0225354. Still further, various embolic filter techniques may be employed as known in the art to protect against emboli production when removing tissue should the selected methodology suggest the need.

In any case, the systems and methods described herein further include the manner in which the implant is delivered after tissue modification alone or modification in combination with resection/removal. In the most basic case (i.e., when the native valve leaflet tissue is modified, but left intact), the prosthetic valve is delivered by first introducing a guidewire into the vascular system and directed to the treatment location by any conventional method, preferably by way of the femoral artery.

In delivering a prosthetic valve assembly as described in commonly-assigned US Publication No. 2005/0203614, after advancing the subject delivery system over the guidewire to the treatment location, its outer sheath is retracted to expose the delivery tube. The gripper provided is then rotated relative to the delivery tube (or the delivery tube rotated relative to the gripper) to cause folded segments of the prosthetic valve to uncurl and to extend radially outward through longitudinal slots of the delivery tube. The delivery tube is then retracted (or the gripper advanced) to cause the prosthetic valve (restrained by the fingers) to advance distally out of the delivery tube. The gripper is then retracted relative to the prosthetic valve, releasing the prosthetic valve into the treatment location. Preferably, the inverted segments then revert to the expanded state, causing the valve to lodge against the internal surface of the body lumen (e.g., the aortic valve root or another biologically acceptable aortic position). Additional expansion of the prosthetic valve may be provided, if needed, by a suitable expansion member, such as an expansion balloon or an expanding mesh member (described elsewhere herein), carried on the delivery catheter or other carrier.

In other methods, different types of prosthetic valves are delivered in deployed. However, certain advantages may, alone, be realized using the above-described type of valve body. By virtue of tissue compliance improvements offered by the techniques described and the structured nature of the implant once expanded, a particularly sound fit between the bodies can be achieved. The ability to accomplish such a result in a percutaneous beating-heart procedure is profound and unprecedented.

Any of the devices described for carrying out the subject methods may be provided in packaged combination for use in executing the method(s). These supply “kits” may further include instructions for use and be packaged in sterile trays or containers as commonly employed for such purposes.

The inventions include methods that may be performed using the subject devices. The methods may all comprise the act of providing such a suitable device. Such provision may be performed by the end user. In other words, the “providing” act merely requires the end user to obtain, access, approach, position, set-up, activate, power-up or otherwise act to provide the requisite device in the subject method. Methods recited herein may be carried out in any order of the recited events which is logically possible, as well as in the recited order of events.

Exemplary embodiments, together with details regarding material selection and manufacture have been set forth above. As for other details of the present invention, these may be appreciated in connection with the above-referenced patents and publications as well as generally know or appreciated by those with skill in the art. For example, one with skill in the art will appreciate that a lubricious coating (e.g., hydrophilic polymers such as polyvinylpyrrolidone-based compositions, fluoropolymers such as tetrafluoroethylene, hydrophilic gel or silicones) may be used in connection with the devices, if desired, to facilitate low friction manipulation or advancement to the treatment site. The same may hold true with respect to method-based aspects in terms of additional acts as commonly or logically employed.

In addition, though the multiple exemplary embodiments have been described, optionally incorporating various features, the inventions are not to be limited to that which is described or indicated as contemplated with respect to each variation. Various changes may be made and equivalents (whether recited herein or not included for the sake of some brevity) may be substituted without departing from the true spirit and scope of the inventions. In addition, where a range of values is provided, it is understood that every intervening value, between the upper and lower limit of that range and any other stated or intervening value in that stated range is encompassed within the inventions.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in the appended claims, the singular forms “a,” “an,” “said,” and “the” include plural referents unless the specifically stated otherwise. In other words, use of the articles allow for “at least one” of the subject item in the description above as well as the claims below. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in the claims shall allow for the inclusion of any additional element—irrespective of whether a given number of elements are enumerated in the claim, or the addition of a feature could be regarded as transforming the nature of an element set forth n the claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of the claim language.

1-67. (canceled)

68. An apparatus for the modification of tissue in a patient, comprising:
an elongate shaft; and
a tissue modification device coupled with the elongate shaft, the tissue modification device configured to mechanically modify target tissue.

69. The apparatus of claim 68, wherein the tissue modification device comprises:
a first rigid element; and
a second rigid element, wherein the first and second rigid elements are configured to transition between a first state, where the rigid elements are in spaced relation to each other, and a second state, where the rigid elements are biased towards each other;
wherein at least one of the first and second rigid elements is coupled to a deflectable arm member.

70. The apparatus of claim 69, wherein the first rigid element is configured to emit an electromagnetic field.

71. The apparatus of claim 69, wherein the first rigid element comprises a textured feature on a surface opposing the second rigid element.

72. The apparatus of claim 68, wherein the tissue modification device comprises at least one weight configured to rotate about the elongate shaft at a radial distance from the shaft.

73. The apparatus of claim 72, wherein the weight is a first weight, the radial distance is a first radial distance and the tissue modification device comprises a second weight located opposite the first weight, the second weight configured to rotate about the elongate shaft at a second radial distance from the shaft, wherein the first and second weights are each coupled to the elongate shaft by way of separate arm members.

74. The apparatus of claim 73, wherein the arm members are coupled to a rotatable hub.

75. The apparatus of claim 73, wherein the first and second radial distance separating the first and second weights from the shaft is variable.

76. The apparatus of claim 73, wherein the tissue modification device further comprises an expandable sheath over the first and second weights.

77. The apparatus of claim 68, wherein the tissue modification device comprises a rotatable weight having a center of mass radially offset from the shaft.

78. The apparatus of claim 68, wherein the tissue modification device is configured to emit ultrasonic energy.

79. An apparatus for the modification of tissue in a patient, comprising:
an elongate shaft; and
a cutting device coupled with the elongate shaft, the cutting device configured to cut target tissue.

80. The apparatus of claim 79, wherein the cutting device comprises a first blade member and a second blade member, each having a sharp outer edge, wherein the first and second blade members are configured to form an arrow-like cutting element.

81. The apparatus of claim 80, wherein the first and second blade members are configured to form the arrow-like cutting element in a first expanded configuration, and are configured to move between the first configuration and a second compressed configuration.

82. The apparatus of claim 79, wherein the cutting device is housable within the elongate shaft.

83. The apparatus of claim 79, wherein the cutting device is configured to sever at least a portion of a valve leaflet.

84. The apparatus of claim 79, wherein the cutting device further comprises a grasping device configured to grasp at least a portion of the target tissue.

85. The apparatus of claim 79, wherein the cutting device comprises:
a flexible wire-like element; and
a cutting element slidably disposed over the wire-like element.

86. The apparatus of claim 85, wherein the flexible wire-like element is configured to conform to the target tissue.

87. The apparatus of claim 86, further comprising a wire band-like element.

88. The apparatus of claim 87, wherein one of the flexible wire-like element and wire band-like element is configured to conform to the target tissue.

89. The apparatus of claim 88, wherein the wire band-like element and wire-like element are configured to pinch tissue therebetween.

90. The apparatus of claim 88, wherein the shape of the band-like element is configured to conform to the shape of the wire-like element.

91. An apparatus for creating a treatment zone within a patient, comprising:
an elongate shaft;
a treatment zone creation device coupled with the elongate shaft, the treatment zone creation device configured to isolate a region of tissue to facilitate the application of a treatment.

92. The apparatus of claim 91, wherein the treatment zone creation device comprises:
a first inflatable toroidal balloon coupled with the elongate shaft;
a second inflatable toroidal balloon coupled with the elongate shaft; and
a third inflatable toroidal balloon coupled with the elongate shaft and configured to be located adjacent to and between the first and second toroidal balloons when the first, second and third balloons are inflated.

93. The apparatus of claim 92, wherein the first and second toroidal balloons are inflatable independent of the third toroidal balloon.

94. The apparatus of claim 93, wherein the third toroidal balloon is configured to evacuate fluids from a region of space within a body lumen.

95. The apparatus of claim 94, wherein the first and second balloons are configured to resist the passage of fluids from an external region into a treatment space adjacent the third balloon when the third balloon is deflated.

96. The apparatus of claim 93, further comprising a treatment device configured to apply energy to a vessel wall in a location opposite the third balloon.
